(19) United States
(12) Patent Application Publication
(10) Pub. No.: US 2014/0236178 A1
(43) Pub. Date: Aug. 21, 2014

(54) MEDICAL TREATMENT TOOL AND MANIPULATOR INCLUDING THE SAME

(71) Applicant: OLYMPUS CORPORATION, Tokyo (JP)

(72) Inventors: Ryoji HYODO, Tokyo (JP); Kosuke KISHI, Tokyo (JP)

(73) Assignee: OLYMPUS CORPORATION, Tokyo (JP)

(21) Appl. No.: 14/266,183

(22) Filed: Apr. 30, 2014

Related U.S. Application Data
(63) Continuation of application No. PCT/JP2012/079443, filed on Nov. 7, 2012.

(30) Foreign Application Priority Data
Nov. 8, 2011 (JP) 2011-244385

(51) Int. Cl. A61B 19/00

(52) U.S. Cl. CPC A61B 19/2203; A61B 2019/2223
USPC 606/130

(57) ABSTRACT

A medical treatment tool includes a treatment section which has a pair of treatment tool pieces that is rotatably supported with respect to a substrate, a manipulating member which rotates the pair of treatment tool pieces, and opens and closes the pair of treatment tool pieces, a link member which has a first end and has a second end, a coupling member which couples the link member and the manipulating member at the second end; a movement indicating part which moves interlocking with a movement of the coupling member in an advance and retraction direction of the manipulating member and is provided so that an amount of movement with respect to the substrate is visually recognized from an outside of the substrate; and a reference index part which is provided at the substrate and refers to a amount of relative movement of the movement indicating part to the substrate.

This application is a continuation application based on PCT/JP2012/079443, filed on Nov. 7, 2012, claiming priority based on Japanese Patent Application No. 2011-244385, filed Nov. 8, 2011. The contents of both the Japanese Patent Application and the PCT Application are incorporated herein by reference.

### BACKGROUND OF THE INVENTION

1. Field of the Invention

The present invention relates to a medical treatment tool and a manipulator including the same.

2. Description of Related Art

Conventionally, medical treatment tools that grasp or press living body tissues, surgical instruments, or the like to perform procedures are used in the medical field.

These medical treatment tools are introduced into body cavities of a patient, and are used for various kinds of procedures by being attached to a manipulator that constitutes, for example, a master slave type medical manipulator system or being inserted through a forceps channel of an endoscope.

In such medical treatment tools, a manipulation force is transmitted to a treatment section at a tip end via links coupled to a manipulating member, such as a wire or a rod, and for example, manipulation, such as the opening and closing of the treatment section is performed in many cases. In this case, an operator visually observes the amount of operation, such as the opening and closing amount of the treatment section, deformation of a body to be treated, sinking of the grasping part into the body to be treated, thereby comprehending the application condition of a force to the body to be treated from the treatment section, or adding or reducing the manipulation force to perform manipulation.

However, in a case where the body to be treated is small and the amount of operation of the treatment section or the deformation amount of the body to be treated are small, it is difficult to comprehend the application condition of the force to the body to be treated. For example, in a case where the treatment section is a grasping part, and the grasping part grasps small members, such as a surgical needle and surgical thread, it is difficult to comprehend the application conditions of a grasping force to the body to be treated even if the grasping part is visually observed.

For this reason, it is known in the medical treatment tools of the related art that graduations or the like for visually recognizing the amount of movement of members, such as a manipulating member that moves corresponding to the application condition of the force from the treatment section is provided on operator's hand side.

As such medical treatment tools of the related art, for example Japanese Patent No. 4350379 describes, as one of the medical treatment tools, a sealing machine for blood vessels that grasps a blood vessel by opening and closing opposed jaw members with links and that gives closing pressure to the blood vessel to perform sealing, and describes a technique that provides a window including graduations or visual marks on a handle manipulated by a user (operator) and that is adapted to be able to observe the compression amount of a coil spring corresponding to a grasping force.

### SUMMARY OF THE INVENTION

A medical treatment tool of a first aspect of the present invention includes a treatment section which has a pair of treatment tool pieces, at least one of the pair of treatment tool pieces being rotatably supported with respect to a substrate; a manipulating member which rotates the pair of treatment tool pieces, and opens and closes the pair of treatment tool pieces by advancing and retracting in an axis direction of the manipulating member with respect to the substrate; a link member which has a first end coupled to the at least one of the pair of treatment tool pieces and has a second end coupled to the manipulating member; a coupling member which couples the link member and the manipulating member at the second end; a movement indicating part which moves interlocking with a movement of the coupling member in an advance and retraction direction of the manipulating member and is provided so that an amount of movement with respect to the substrate is visually recognized from an outside of the substrate; and a reference index part which is provided at the substrate and refers to a amount of relative movement of the movement indicating part to the substrate.

In a medical treatment tool of a second aspect of the present invention, in the first aspect, the substrate may be provided with a through hole portion, the movement indicating part may be provided at the coupling member that is movably inserted through the through hole portion, and the reference index part may be a reference scale provided around the through hole portion, or on the through hole portion.

In a medical treatment tool of a third aspect of the present invention, in the first or second aspect, the reference index part may have a shielding part that covers a portion of the surface of the substrate, the movement indicating part may have an indicating member that advances and retract from an inside of the shielding part to an outside and that interlocks with the movement of the coupling member, and the amount of movement of the movement indicating part may be indicated depending on an amount of an advance and retraction of the indicating member from the shielding part.

In a medical treatment tool of a fourth aspect of the present invention, in any one of the first to third aspects, the reference index part may include a closed state starting position reference index that allows a position of the movement indicating part when a state where the pair of treatment tool pieces is closed is started to be referred to, and a closing force reference index that allows a change in the position of the movement indicating part corresponding to an increase in a closing force between the pair of treatment tool pieces in a state where the pair of treatment tool pieces is closed to be referred to.

In a medical treatment tool of a fifth aspect of the present invention, in any one of the first to fourth aspects, an advance and retraction axis of the second end may be parallel to the axis of the manipulating member, a distance between the advance and retraction axis of the second end and the first end may be shorter than the length of the link member, and a length when a line segment connecting the second end and a rotation center of the pair of treatment tool pieces is projected on the advance and retraction axis may be shorter than a length when a line segment connecting the first end and the rotation center is projected on the advance and retraction axis.

A manipulator of a sixth aspect of the present invention includes the medical treatment tool of the present invention in any one of the first to fifth aspects.

### BRIEF DESCRIPTION OF THE DRAWINGS

FIG. 1 is a schematic view showing an example of the configuration of a medical manipulator system to which a medical treatment tool of a first embodiment of the present invention is applied.

FIG. 2 is a schematic front view showing a tip end of the medical treatment tool of the first embodiment of the present invention.

FIG. 3 is a schematic cross-sectional view showing the inside of the tip end of the medical treatment tool of the first embodiment of the present invention.

FIG. 4 is a schematic cross-sectional view showing a state where a treatment section of the medical treatment tool of the first embodiment of the present invention is opened.

FIGS. 5A and 5B are schematic front views showing a state that the treatment section of the medical treatment tool of the first embodiment of the present invention is opened and a state where the treatment section is closed and a grasping force is increased.

FIG. 6 is a schematic view for describing the action of a grasping force increase in a toggle mechanism.

FIG. 7A is a schematic front view showing a state where a treatment section of a medical treatment tool of a first modified example of the first embodiment of the present invention is closed.

FIG. 7B is a schematic front view showing a state where the treatment section of the medical treatment tool of the first modified example of the first embodiment of the present invention is opened.

FIG. 8A is a schematic front view showing a state where a treatment section of a medical treatment tool of a second modified example of the first embodiment of the present invention is opened.

FIG. 8B is a schematic front view showing a state where the treatment section of the medical treatment tool of the second modified example of the first embodiment of the present invention is closed.

FIG. 9 is a schematic front view showing a treatment section of a medical treatment tool of a third modified example of the first embodiment of the present invention.

FIG. 10A is a schematic front view showing a treatment section of a medical treatment tool of a fourth modified example of the first embodiment of the present invention.

FIG. 10B is a side view seen at A of FIG. 10A.

FIG. 11 is a schematic front view showing a treatment section of a medical treatment tool of a fifth modified example of the first embodiment of the present invention.

FIG. 12A is a schematic partial cross-sectional view showing a state where a treatment section of a medical treatment tool of a second embodiment of the present invention is opened.

FIG. 12B is a schematic partial cross-sectional view showing a state where the treatment section of the medical treatment tool of the second embodiment of the present invention is closed.

FIG. 12C is a schematic partial cross-sectional view showing a state where the treatment section of the medical treatment tool of the second embodiment of the present invention is closed and a grasping force is increased.

FIG. 13A is a schematic front view showing a treatment section of a medical treatment tool of a third embodiment of the present invention.

FIG. 13B is a schematic cross-sectional view showing the treatment section of the medical treatment tool of the third embodiment of the present invention.

### DETAILED DESCRIPTION OF THE INVENTION

Embodiments of the present invention will be described below with reference to the accompanying drawings. In all the drawings, even if embodiments are different, the same reference numerals will be given to the same or equivalent members, and common description will be omitted.

### First Embodiment

Although a first embodiment of the present invention will be described below, an example of a medical treatment tool (hereinafter simply referred to as “treatment tool”) of the present embodiment and a medical manipulator system to which the manipulator is applied will be described.

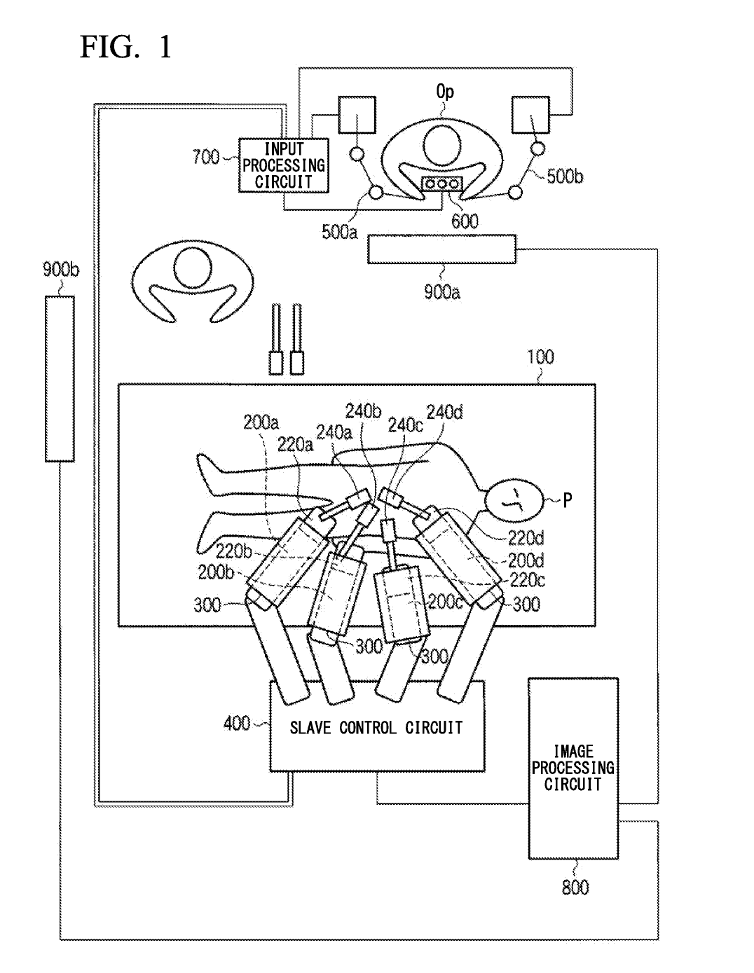

FIG. 1 is a schematic view showing an example of the configuration of a medical manipulator system to which a medical treatment tool of a first embodiment of the present invention is applied.

An example of a master slave type medical manipulator system is shown in FIG. 1. The master slave type medical manipulator system is a system that has two kinds of arms including a master arm and a slave arm and remotely controls the slave arm so as to follow the operation of the master arm. The manipulator of the first embodiment of the present invention can be applied as this slave arm.

The medical manipulator system shown in FIG. 1 has a surgical table 100, slave arms 200a, 200b, 200c, and 200d (manipulators), a slave control circuit 400, master arms 500a and 500b, a manipulating unit 600, an input processing circuit 700, an image processing circuit 800, a display 900a for an operator, and a display 900b for an assistant.

Hereinafter, in order to simplify description, symbols “Xa, Xb, Xz” in an alphabetical order may be expressed like “Xa to Xz”. For example, the “slave arms 200a, 200b, 200c, and 200d” may be expressed as “slave arms 200a to 200d”.

The surgical table 100 is a table on which a patient P who is a target to be observed and treated lies down. The plurality of slave arms 200a to 200d is installed in the vicinity of the surgical table 100. In addition, the slave arms 200a to 200d may be installed on the surgical table 100.

The slave arms 200a to 200d have a plurality of multi-degree-of-freedom joints, respectively, and bends the respective multi-degree-of-freedom joints, thereby positioning a treatment tool to be mounted on the tip ends (the side that faces the body cavity of the patient P) of the slave arms 200a to 200d with respect to the patient P lying on the surgical table 100. The respective multi-degree-of-freedom joints are individually driven by power units (not shown). As the power units, for example, motors (servo motors) having a servo mechanism including an incremental encoder, a decelerator, or the like can be used, and the motion control of the power unit is performed by the slave control circuit 400.

The slave arms 200a to 200d have a plurality of power units for driving mounted treatment tools 240a to 240d (not shown). As the power units, for example, servo motors can also be used, and the motion control of the power units is also performed by the slave control circuit 400.

In a case where the power units of the slave arms 200a to 200d are driven, the driving amounts of the power units are detected by position detectors. Detection signals from the position detectors are input to the slave control circuit 400, and the driving amounts of the slave arms 200a to 200d are detected in the slave control circuit 400 by the detection signals.

Power transmission adapters 220a, 220b, 220c, and 220d for operation (hereinafter simply referred to as “adapters”) are interposed between the slave arms 200a to 200d and the treatment tools 240a to 240d to connect the slave arms 200a to 200d and the treatment tools 240a to 240d, respectively. The adapters 220a to 220d each have a linear driving mechanism, and are configured so as to transmit the power generated in the power units of the corresponding slave arm to the corresponding treatment tools by linear driving motion.

The slave control circuit 400 is configured to have, for example, a CPU, a memory, or the like. The slave control circuit 400 stores a predetermined program for performing the control of the slave arms 200a to 200d, and controls the operation of the slave arms 200a to 200d or the treatment tools 240a to 240d according to a control signal from the input processing circuit 700. That is, the slave control circuit 400 specifies a slave arm (or treatment tool) that is a manipulation target of a master arm manipulated by the operator Op on the basis of the control signal from the input processing circuit 700, and computes a driving amount that is required to cause the specified slave arm to make a movement corresponding to the amount of manipulation of the master arm by the operator Op.

Also, the slave control circuit 400 controls the operation of a slave arm or the like that is a manipulation target of the master arm according to the computed driving amount. In this case, the slave control circuit 400 inputs a driving signal to a corresponding slave arm, and controls the magnitude or polarity of the driving signal so that the driving amount of the slave arm that is a manipulation target becomes a target driving amount according to a detection signal input from a position detector of a power unit according to the operation of the corresponding slave arm.

The master arms 500a and 500b are constituted by a plurality of link mechanisms. Respective links that constitute the link mechanisms is provided with, for example, position detectors, such as an incremental encoder. By detecting the operation of the respective links using the position detectors, the amount of manipulations of the master arms 500a and 500b are detected in the input processing circuit 700.

The medical manipulator system of FIG. 1 needs to manipulate four slave arms using two master arms 500a and 500b and appropriately switch the slave arms that are manipulation targets of the master arms. Such switching is performed, for example, by the manipulation of the manipulating unit 600 by the operator Op. Of course, such a change are unnecessary if manipulation targets has the correspondence of 1 to 1 by making the number of master arms and the number of slave arms the same.

The manipulating unit 600 has switching buttons for switching the slave arms that are manipulation targets of the master arms 500a and 500b, and various kinds of manipulating members, such as a scaling changing switch that changes the operation ratio of a slave and a master, and a foot switch for urgently stopping the system. In a case where a certain manipulating member that constitutes the manipulating unit 600 is manipulated by the operator Op, a manipulation signal according to the manipulation of the corresponding manipulating member is input to the input processing circuit 700 from the manipulating unit 600.

The input processing circuit 700 analyzes the manipulation signals from the master arms 500a and 500b and the manipulation signal from the manipulating unit 600, and generates a control signal for controlling the medical manipulator system according to an analysis result of the manipulation signal, to input the control signal to the slave control circuit 400.

The image processing circuit 800 performs various kinds of image processing for displaying an image signal input from the slave control circuit 400, to generate image data for display in the display 900a for an operator and the display 900b for an assistant. The display 900a for an operator and the display 900b for an assistant are constituted by, for example, liquid crystal displays, and displays an image based on the image data generated in the image processing circuit 800 according to an image signal acquired via the observation instrument.

In the medical manipulator system configured as described above, if the operator Op manipulates the master arms 500a and 500b, a corresponding slave arm and a treatment tool attached to this slave arm operate in response to the movement of the master arms 500a and 500b. Thereby, a desired procedure can be performed on patient P.

Next, the medical treatment tool of the present embodiment will be described.

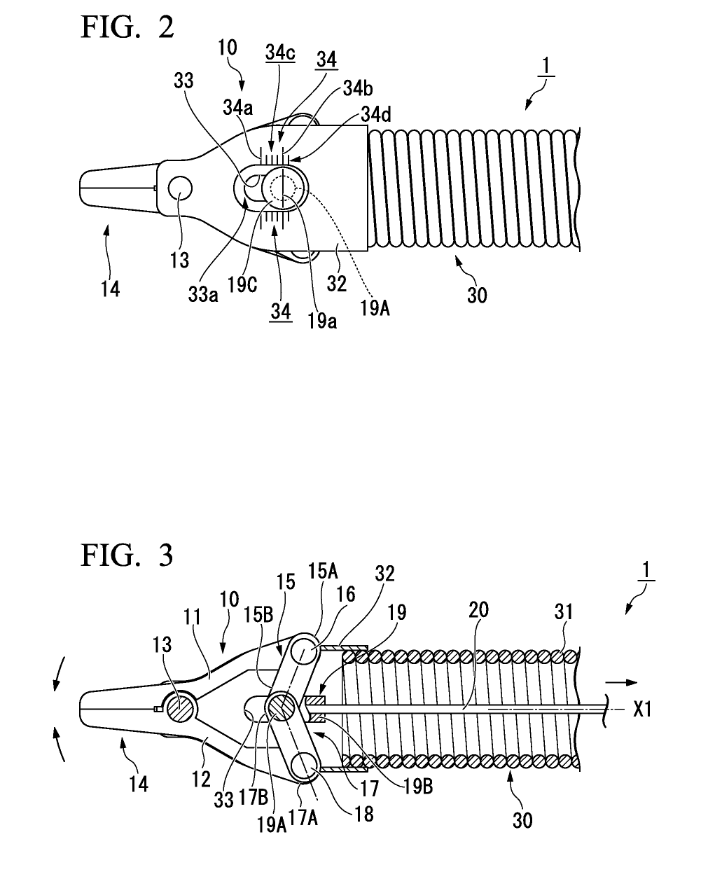

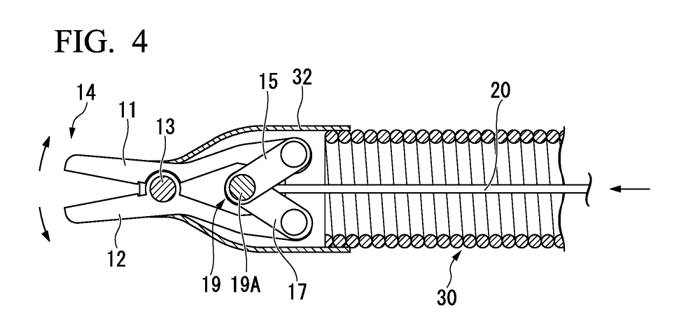

FIG. 2 is a schematic front view showing a tip end of the medical treatment tool of the first embodiment of the present invention. FIG. 3 is a schematic cross-sectional view showing the inside of the tip end of the medical treatment tool of the first embodiment of the present invention. FIG. 4 is a schematic cross-sectional view showing a state where the treatment section of the medical treatment tool of the first embodiment of the present invention is opened.

A treatment tool 1 (medical treatment tool) can be mounted on the slave arms 200a to 200d as the above-described treatment tools 240a to 240d. The treatment tool 1, as shown in FIGS. 2 and 3, includes a treatment section 10 for performing various kinds of treatment, a manipulating member 20 for manipulating the treatment section 10, and a sheath part 30 through which the manipulating member 20 is inserted.

The treatment section 10, as shown in FIG. 3, includes a pair of forceps pieces (treatment tool pieces) including the first forceps piece 11 and a second forceps piece 12. The first forceps piece 11 and the second forceps piece 12 are mutually rotatably coupled by a forceps rotation shaft 13, and a region closer to the tip end side than the forceps rotation shaft 13 is a grasping part 14 that is opened and closed to grasp objects, such as a body tissue or a surgical instrument.

On the base end side (the side opposite of the grasping part 14) of the first forceps piece 11, a tip end 15A (first end) of a link member 15 is rotatably coupled to the first forceps piece 11 via a link rotation shaft 16. Similarly, on the base end side of the second forceps piece 12, a tip end 17A (first end) of a link member 17 is rotatably coupled to the second forceps piece 12 via a link rotation shaft 18.

Both the central axes of the link rotation shafts 16 and 18 are parallel to the central axis of the forceps rotation shaft 13. Additionally, the respective tip ends 15A and 17A of the respective link members 15 and 17 are coupled closer to the manipulating member 20 side than the forceps rotation shaft 13.

In the present embodiment, the positions of the tip ends 15A and 17A that are ends of the link members indicate the positions of the rotation centers of the link rotation shafts 16 and 18 that are the positions of the rotation centers of the tip ends 15A and 17A, unless explicitly stated.

Additionally, the positions of ends of other link members to be described below similarly indicate the positions of the rotation centers of rotation shafts coupled to the ends.

Base ends 15B and 17B (second ends) of the link members 15 and 17 are rotatably connected to a connecting member 19 (coupling member) via a connection rotation shaft 19A. The central axis of the connection rotation shaft 19A is parallel to the respective central axes of the forceps rotation shaft 13 and the link rotation shafts 16 and 18, and the respective link members 15 and 17 are rotatable relative to the connecting member 19.

The connecting member 19 is formed from metal or the like and has a connection rotation shaft 19A that rotationally supports the base ends 15B and 17B on the tip end side thereof. The base end of the connecting member 19 is a manipulating member connecting portion 19B that is formed in a substantially cylindrical shape, and a tip end of the manipulating member 20 is inserted into the manipulating member connecting portion 19B and is integrally connected thereto by welding, adhesion, caulking, or the like.

The manipulating member 20 is an elongate member and has such a degree of rigidity that the advancing and retracting manipulation in the base end (not shown) can be transmitted to the tip end. The manipulating member 20 is connected integrally with the connecting member 19 on the base end side of the treatment section 10 as the tip end thereof is inserted into the manipulating member connecting portion 19B of the connecting member 19.

The sheath part 30 includes a sheath 31 that is formed in a tubular shape, and the manipulating member 20 is inserted into the sheath 31 so as to be able to advance and retract. In the present embodiment, a well-known coiled sheath having flexibility is used as the sheath 31.

A cover member 32 (substrate) formed from metal or the like is attached to a tip end of a sheath 31. The forceps rotation shaft 13 is fixed to the cover member 32. That is, the first forceps piece 11 and the second forceps piece 12 are rotatably supported with respect to the cover member 32, and the forceps rotation shaft 13 is fixed so as not to move with respect to the sheath part 30.

Additionally, the cover member 32 is formed with an elongate hole 33 (through hole) that extends in a direction along the axis X1 of the manipulating member 20. The connection rotation shaft 19A is inserted through the elongate hole 33 without rattling, and if the manipulating member 20 is attracted and retracted in the direction of the axis X1, the connection rotation shaft 19A can smoothly move in the direction along the longitudinal direction of the elongate hole 33 interlocking with the movement of the manipulating member 20. Thereby, the base ends 15B and 17B of the respective link members coupled with the connection rotation shaft 19A also move along the elongate hole 33.

Hereinafter, a straight line formed by the movement track of a point on the central axis of the connection rotation shaft 19A during advance and retraction of the manipulating member 20 is referred to as “advance and retraction axis of the base ends”. That is, in the present embodiment, a symmetry axis along the longitudinal direction of the elongate hole 33 and the advance and retraction axis of the base ends 15B and 17B are parallel to each other.

Although the elongate hole 33 may be provided only on one side of the cover member 32, the elongate holes are provided in two places in the present embodiment from the positional relationship of penetrating in the depth direction of FIG. 3. For this reason, the connection rotation shaft 19A is inserted through the two elongate holes 33.

A step hole portion 33a that has an elongate hole shape slightly larger than each elongate hole 33 and is depressed inward from the external surface of the cover member 32 is formed around each elongate hole 33.

Each step hole portion 33a is provided in a size such that disc-shaped movement indicating parts 19C that are formed coaxially with the connection rotation shaft 19A at both ends of the connection rotation shaft 19A can be accommodated. In the present embodiment, as an example, each step hole portion has a slightly larger width than the external diameter of the movement indicating part 19C and almost the same thickness as the thickness of the movement indicating part 19C.

In addition, the movement indicating part 19C may be made of member that is separate from the connection rotation shaft 19A and may be fixed to the connection rotation shaft 19A. The fixing method is not particularly limited, and for example, screwing, screw fastening, welding, caulking, or the like can be appropriately adopted.

Additionally, a straight indicating mark 19a that extends in a direction orthogonal to the movement direction of the connection rotation shaft 19A is formed in the surface of the movement indicating part 19C that is viewed from the outside of the cover member 32.

As the method of forming the indicating mark 19a, an appropriate forming method may be adopted. For example, examples printing, stamping, laser beam machining, etching, and inlaying may be mentioned.

Additionally, the indicating mark 19a may be formed by providing straight concave groove portions or projection portions in the movement indicating part 19C. Additionally, if the correspondence to the graduation lines 34 to be described below can be easily and visually recognized, the indicating mark 19a is not limited to a straight line, and appropriate figures, such as arrow marks, triangular marks, cross marks, and the like can be adopted. Additionally, a configuration may be adopted in which the outer shape of the movement indicating part 19C itself also serves as the indicating mark 19a.

Additionally, as the indicating mark 19a, for example, in a case where the movement indicating part 19C is fixed to the connection rotation shaft 19A with screws, shapes, such as minus holes or cross holes, which are formed in screw heads, may also serve as the indicating mark 19a such that these figures are viewed.

Additionally, a configuration may be adopted in which the outer shape of the movement indicating part 19C itself can point positions by providing projection portions, cutout portions, and acute portions as the outer shape of the movement indicating part 19C.

As shown in FIG. 2, graduation lines 34 (reference index part) are formed on the cover member 32 around each step hole portion 33a.

The graduation lines 34 are reference scales in order to refer to the amount of relative movement of the movement indicating part 19C to the cover member 32.

As the graduation lines 34, in the present embodiment, as an example, parallel line groups, which extend in the direction orthogonal to the longitudinal direction of the step hole portion 33a and are formed at regular pitches in the longitudinal direction, is provided so as to make a pair at positions that face each other in the lateral direction of the step hole portion 33a.

Additionally, the respective graduation lines 34 are constituted, as an example, by a maximum open position graduation 34a, a basis closed position graduation 34b (closed state starting position reference index), an open position auxiliary graduation 34c, and a grasping force reference auxiliary graduation 34d (closing force reference index).

The maximum open position graduation 34a is a graduation that shows the position of the indicating mark 19a when the connection rotation shaft 19A has moved to the tip end side (grasping part 14 side) most within the elongate hole 33.

The basis closed position graduation 34b is a graduation corresponding to the position of an indicating mark 19a when the grasping part 14 is exactly closed (when the closed state of the grasping part 14 is started). In other words, the basis closed position graduation 34b refers to the position of the movement indicating part 19C when the closed state of the grasping part 14 is started. Additionally, in the present embodiment, the basis closed position graduation 34b includes a line segment that is parallel to the maximum open position graduation 34a and has equal length.

The open position auxiliary graduation 34c is an auxiliary graduation that divides the maximum open position graduation 34a and the basis closed position graduation 34b equally so that the position between the maximum open position graduation 34a and the basis closed position graduation 34b can be referred to. In the present embodiment, the open position auxiliary graduation includes mutually parallel line segments that divide the maximum open position graduation 34a and the basis closed position graduation 34b equally and are shorter than the maximum open position graduation 34a and the basis closed position graduation 34b.

When the indicating mark 19a has moved closer to the base end side than the basis closed position graduation 34b, the grasping force reference auxiliary graduation 34d refers to the position of the indicating mark 19a corresponding to an increase in the closing force of the grasping part 14. Additionally, the grasping force reference auxiliary graduation 34d is a parallel line segment that is shorter than the basis closed position graduation 34b, similarly to the open position auxiliary graduation 34c. In the present embodiment, as an example, a line segment that indicates the position when the connection rotation shaft 19A has moved to the base end side (the side opposite to the grasping part 14) most within the elongate hole 33 is formed on the cover member 32.

As the method of forming the graduation lines 34, the same forming method as the indicating mark 19a can be adopted.

With respect to the operation when the treatment tool 1 configured as described above is used, a case where the treatment tool is attached to one of the above-described slave arms 200a to 200d will be described as an example.

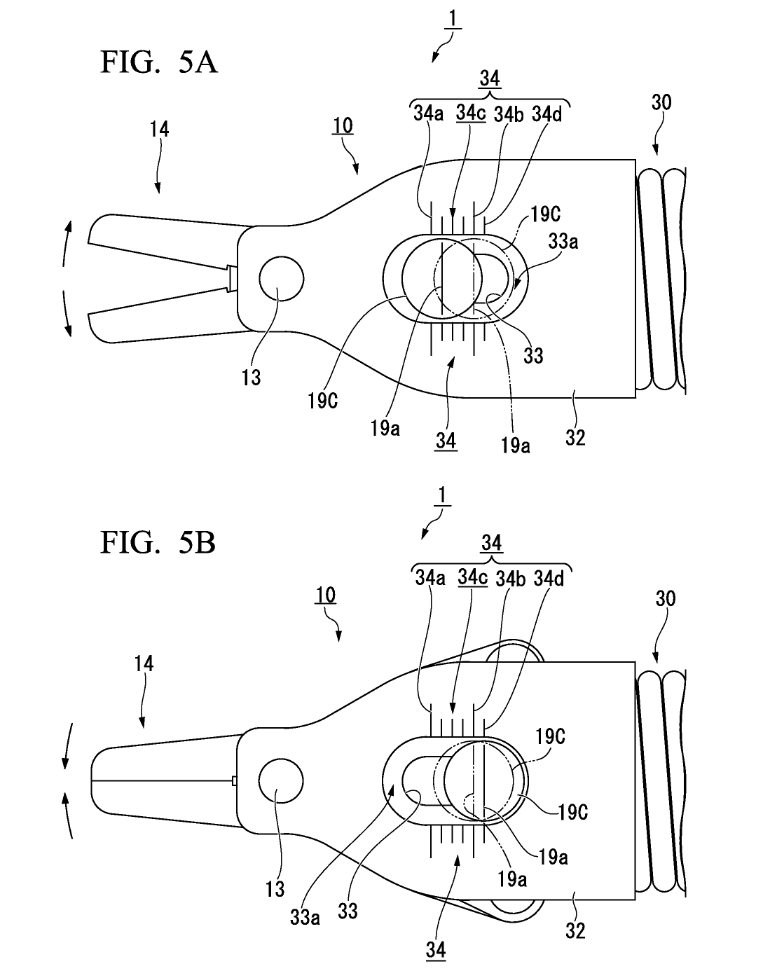

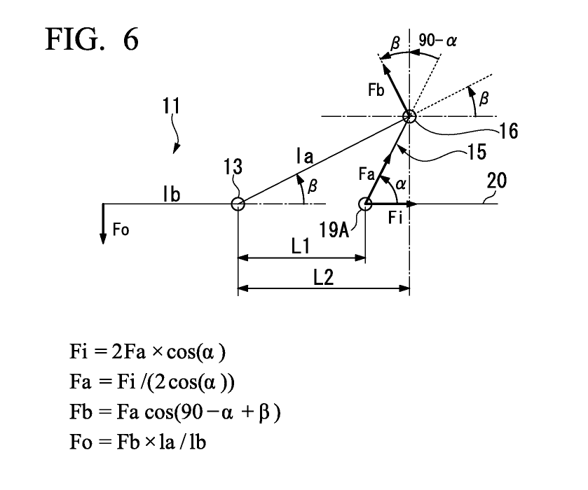

FIG. 5A is a schematic front view showing a state where the treatment section of the medical treatment tool of the first embodiment of the present invention is opened. FIG. 5B is a schematic front view showing a state where the treatment section of the medical treatment tool of the first embodiment of the present invention is closed and a grasping force is increased. FIG. 6 is a schematic view for describing the action of a grasping force increase in a toggle mechanism.

First, the operator Op mounts the treatment tool 1 on a desired slave arm, and connects the base end of the manipulating member 20 to an adapter of the slave arm.

The operator Op comprehends the positional relationship between the treatment section 10 of the treatment tool 1 and a target that the grasping g part 14 of the treatment section 10 should grasp by viewing an image displayed on the display 900a for an operator, and manipulates a master arm that manipulates the slave arm to which the treatment tool 1 is connected.

If the operator Op performs predetermined manipulation on the corresponding master arm, a power unit of the slave arm is driven via the slave control circuit 400a. The power generated in the power unit is converted into a linear driving motion via the adapter, and the manipulating member 20 is advanced and retracted in the direction of the axis X1 by a linear driving motion.

If the manipulating member 20 is advanced, the connecting member 19 connected to the manipulating member 20 advances with respect to the sheath part 30. However, since the forceps rotation shaft 13 is fixed to the cover member 32, the forceps rotation shaft does not advance with respect to the sheath part 30. Thereby, the connection rotation shaft 19A approaches the forceps rotation shaft 13, and the link members 15 and 17 rotate with respect to the first forceps piece 11, the second forceps piece 12, and the connecting member 19. As a result, the first forceps piece 11 and the second forceps piece 12 rotate around the forceps rotation shaft 13, and as shown in FIG. 4, the grasping part 14 is opened.

At this time, according to the amount of movement of the connection rotation shaft 19A, as shown by a solid line FIG. 5A, the movement indicating part 19C moves within the step hole portion 33a. This situation is displayed on the display 900a for an operator that holds a manipulation target and the treatment section 10 in a visual field region.

For this reason, the operator Op can refer to the graduation lines 34 by viewing the display 900a for an operator to thereby comprehend the amount of relative movement or movement position of the indicating mark 19a of the movement indicating part 19C.

For example, in FIG. 5A, the indicating mark 19a is located at the position of the open position auxiliary graduation 34c nearest to the maximum open position graduation 34a. Therefore, it can be easily seen that the grasping part 14 is opened by about ¾ of the maximum opening angle of the grasping part 14.

Thereby, the operator Op can perform determination on, for example, whether the grasping part 14 should be more greatly opened in order to continue grasping manipulation and whether the opening angle of the grasping part 14 should be reduced in order to more reliably grasp an object that is already grasped.

In that case, since the operator Op can comprehend the opening angle of the grasping part 14 without keeping his/her eyes off the display 900a for an operator on which the manipulation target and the grasping part 14 are displayed, manipulation can be smoothly continued while monitoring the manipulation target even by delicate manipulation.

Additionally, for example, if the operator Op performs the manipulation of retracting the manipulating member 20, the connection rotation shaft 19A separates from the forceps rotation shaft 13, and the grasping part 14 is closed by the operation reverse to the above-described one. Accordingly, it is possible to perform such as desired procedures, such as advancing and retracting the manipulating member 20 in the direction of the axis X1 with respect to the cover member 32, thereby opening and closing the grasping part 14 to grasp a target tissue or grasp tools required for the treatment of a curved needle, suture thread, or the like.

In the present embodiment, as shown in FIG. 3, the first forceps piece 11 and the second forceps piece 12 comes into contact with each other closer to the tip end side than the forceps rotation shaft 13, and the connection rotation shaft 19A is located closer to the tip end side than the respective link rotation shafts 16 and 18 in a state where the grasping part 14 is closed. That is, the base ends 15B and 17B of the respective link members 15 and 17 are located closer to the tip end side than the tip ends 15A and 17A.

Additionally, the distance between the advance and retraction axis of the base ends 15B and 17B of the respective link members 15 and 17 and the link rotation shaft 16 is shorter than the length of the link member 15. Similarly, the distance between the advance and retraction axis of the base ends and the link rotation shaft 18 is shorter than the length of the link member 17.

Moreover, as shown in FIG. 6, the length L1 when a line segment connecting the rotation center (central axis of the forceps rotation shaft 13) of a pair of treatment tool pieces and the position (the position of the rotation center of the connection rotation shaft 19A) of the base ends of the link members is projected on the advance and retraction axis of the base ends is set so as to become shorter than the length L2 when a line segment connecting the rotation center and the position (the position of the rotation center of the link rotation shaft 16) of the tip ends of the link members is projected on the advance and retraction axis of the base ends. In addition, the details of FIG. 6 will be described below.

From such a configuration, the connecting member 19, the respective link members 15 and 17, and the first forceps piece 11 and the second forceps piece 12 function as a so-called toggle mechanism. Accordingly, the respective link members 15 and 17, and the first forceps piece 11 and the second forceps piece 12 are elastically deformed by further performing manipulation input on the manipulating member 20 in the retracting direction from a state where the grasping part 14 is closed. The base ends 15B and 17B of the respective link members coupled with the connecting member 19 move along the axis X1 of the manipulating member 20. That is, the advance and retraction axis of the base ends are parallel to the axis X1 (including substantially parallel). At this time, although the appearance of the grasping part 14 when being closed hardly changes, a grasping force generated in the grasping part 14 increases.

The details of the above-described grasping force increase will be described. The first forceps piece 11, the forceps rotation shaft 13, the link member 15, the link rotation shaft 16, the connection rotation shaft 19A, and the manipulating member 20 are schematically shown in FIG. 6. As shown in FIG. 6, if a manipulation input Fi is made to act on the manipulating member 20 and the manipulation member is retracted, the connection rotation shaft 19A retracts and the angle α formed by the advance and retraction axis of the base ends and the link member 15 on the base end side becomes large, and a force Fb of moving the link rotation shaft 16 in a direction in which the link rotation shaft 16 is separated from the axis of the manipulating member 20 is generated. The force Fb acts so as to rotate the first forceps piece 11 and the second forceps piece 12 around the forceps rotation shaft 13, and finally output Fo is generated in the grasping part.

1a and 1b shown in FIG. 6 represents the length of the region closer to the tip end side than the forceps rotation shaft 13 and the length of the region closer to the base end side than the forceps rotation shaft 13 in the respective forceps pieces, and the angle 13 represents an angle formed by the advance and retraction axis of the connection rotation shaft 19A and a straight line connecting the forceps rotation shaft 13 and the link rotation shaft 16. Additionally, although the second forceps piece 12 or the like is not shown, the output Fo is similarly generated.

The magnitude of the output Fo that is generated and acts on the grasping part 14 in the first forceps piece 11 and the second forceps piece 12 is expressed by the following Formula (1). Here, α and β are the angles expressed in units of degrees (°).

$$\begin{matrix}[ Formula 1 ] & \\ Fo = \frac{Fi \cdot 1 a \cdot cos ( 90 - α + β )}{2 \cdot 1 b \cdot cos ( α )} & ( 1 )\end{matrix}$$

Accordingly, the output Fo becomes exponentially larger as the angle c approaches 90 degrees. The output Fo can be theoretically made infinite. However, actually, if the output Fo becomes more than a predetermined magnitude, the respective link members 15 and 17 or the first forceps piece 11 and the second forceps piece 12 deform plastically. Therefore, the upper limit of the grasping force is defined by the yield stress of these members.

The shape of the elongate hole 33 formed in the cover member 32 and the dimension of the manipulating member 20 in the advance and retraction direction are set in consideration of the shape of an object to be grasped and the above yield stress. Therefore, even if the manipulating member 20 is retracted until the connection rotation shaft 19A comes into contact with the base end of the elongate hole 33 in a state where an object is grasped, the respective link members 15 and 17, the first forceps piece 11 and the second forceps piece 12 do not cause plastic deformation. That is, the elongate hole 33 functions as a guide that moves the base ends of the respective link members coupled to the connection rotation shaft 19A along the direction of the axis X1 of the manipulating member 20, and functions also as a stopper that comes into contact with the connection rotation shaft 19A to regulate the maximum retraction amount of the manipulating member 20.

In such closing manipulation, the magnitude of the grasping force changes markedly depending on the retraction amount of the manipulating member 20. Therefore, it is important to exactly comprehend the retraction amount of the manipulating member 20.

Additionally, in the closing manipulation, a grasp target is grasped by the grasping part 14. Therefore, for example, in the case of a grasp target that does not easily deform, such as a surgical needle or a surgical instrument, the closing amount of the grasping part 14 hardly changes even if the closing manipulation is performed. For this reason, it is difficult to comprehend the application condition of a force to a grasp target only by observing the opening angle of the grasping part 14.

In contrast, in the present embodiment, since the operator Op can comprehend the amount of movement of the indicating mark 19a with reference to the graduation lines 34 during manipulation while viewing the display 900a for an operator, the operator can easily comprehend the degree of an increase in the grasping force during closing manipulation.

For example, in a state where the grasping part 14 is exactly closed, the indicating mark 19a of the movement indicating part 19C, as shown by a two-dot chain line in FIG. 5B, is at the position of the basis closed position graduation 34b, and the grasping force of the grasping part 14 is 0 at this time.

If the operator Op retracts the manipulating member 20 from this state, the grasping force of the grasping part 14 increases steeply, and if the movement indicating part 19C retracts at the maximum and the indicating mark 19a coincides with the grasping force reference auxiliary graduation 34d as shown by a solid line in FIG. 5B, the grasping force becomes maximum.

In a case where the grasping part 14 is not grasping a grasp target in this way, the position of the basis closed position graduation 34b becomes a basis of the grasping force. However, in a case where the grasp target is grasped, grasping is started and the position of the indicating mark 19a in a grasp state where the movement of the grasping g part 14 has stopped, the grasping force becomes a basis position of 0.

For example, when a high-hardness grasp target like a surgical needle is grasped, supposing the indicating mark 19a indicates the open position auxiliary graduation 34c shifted by one graduation from the basis closed position graduation 34b, the grasping force as shown by the above Formula (1) increases while one graduation moves on the graduation lines 34 from the open position auxiliary graduation 34c.

Accordingly, in a case where it is known that a grasping force suitable for a grasp target is, for example, ½ graduation, the operator Op can easily retract the manipulating member 20 to a position where the suitable grasping force is obtained by observing the position of the indicating mark 19a while performing manipulation.

As described above, according to the treatment tool 1 of the present embodiment, a toggle mechanism is constituted by the first forceps piece 11, the second forceps piece 12, the respective link members 15 and 17, and the connecting member 19 of the treatment section 10. Therefore, by performing the manipulation input of further retracting the manipulating member 20 from a state where the grasping part 14 is closed, a grasping force generated in the grasping part can be efficiently increased even if a manipulation input is relatively small.

Additionally, since the treatment section 10 are provided with the movement indicating part 19C and the graduation lines 34, the opening and closing amount or force application condition of the grasping part 14 of the treatment section 10 can be easily comprehended while viewing the movement indicating part 19C and the graduation lines 34 during manipulation.

In that case, the movement indicating part 19C is fixed to the connection rotation shaft 19A that is manipulated by the manipulating member 20 to drive the toggle mechanism. For this reason, the amount of movement of the movement indicating part 19C from a state where the grasping part 14 is closed or a state where the movement of the grasping part 14 in the closed direction has stopped becomes larger compared to, for example, a case where the movement indicating part is provided at the tip end of the manipulating member that manipulates a pantagraph mechanism. For this reason, a change in the grasping force can be more finely comprehended depending on the amount of movement of the movement indicating part 19C.

Additionally, although the reaction force of the output Fo acts on the connection rotation shaft 19A on which the manipulation input Fi acts so that the connection rotation shaft is separated from the axis of the manipulating member 20, the respective link members 15 and 17 of the present embodiment are arranged substantially bilaterally symmetrically with respect to the movement direction of the connection rotation shaft 19A. Due to the substantially symmetrical arrangement as described above, the connection rotation shaft 19A is located almost in the middle of the link rotation shafts 16 and 18 in the opening and closing direction of the grasping part 14, and the reaction force of the output Fo that acts on each link rotation shaft acts in an approximately opposite direction. As a result, the reaction force is cancelled out and becomes almost zero.

Accordingly, the connection rotation shaft 19A inserted through the elongate hole 33 is not strongly pressed against the inner surface of the elongate hole 33, and generation of large friction is suppressed.

### First Modified Example

Next, a first modified example of the present embodiment will be described.

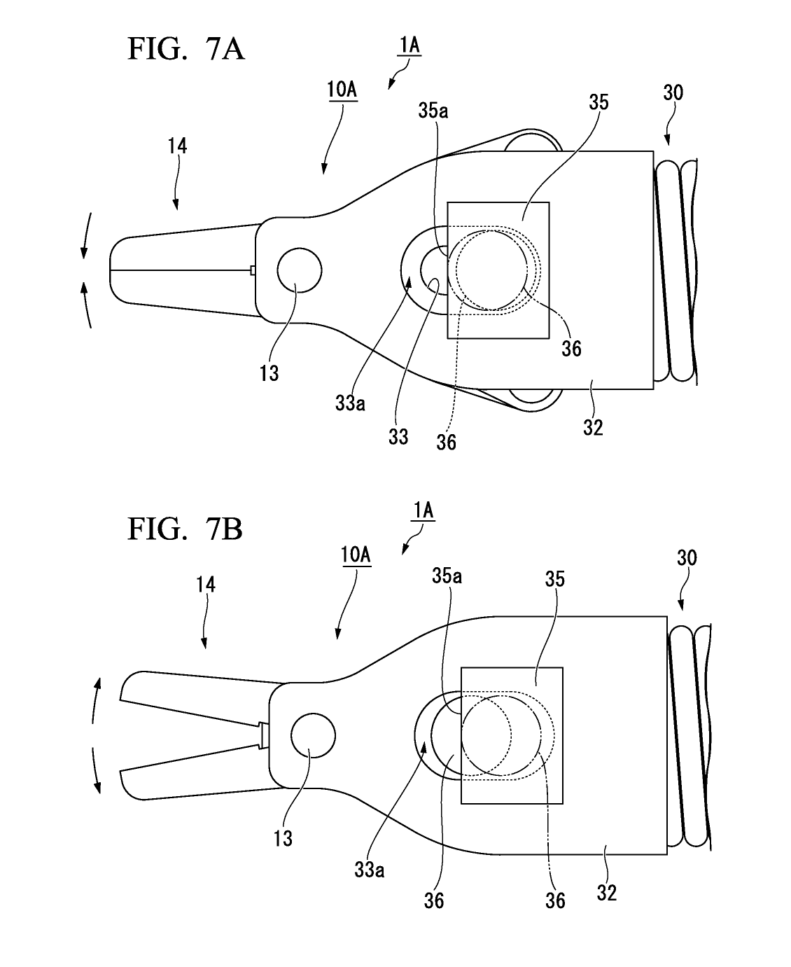

FIG. 7A shows a state where a treatment section of a medical treatment tool of the first modified example of the first embodiment of the present invention is closed. FIG. 7B is a schematic front view showing a state where the treatment section of the medical treatment tool of the first modified example of the first embodiment of the present invention is opened.

A treatment tool 1A (medical treatment tool) of the present modified example, as shown in FIG. 7A, includes a treatment section 10A instead of the treatment section 10 of the treatment tool 1 of the first embodiment, and can be mounted on the slave arms 200a to 200d and used as the treatment tools 240a to 240d of the medical manipulator system shown in FIG. 1 similarly to the first embodiment.

Hereinafter, differences from the first embodiment will mainly be described.

The treatment section 10A includes a movement indicating part 36 (indicating member) and a shielding part 35 (reference index part) instead of the movement indicating part 19C and the graduation lines 34 of the treatment section 10 of the first embodiment.

In the movement indicating part 36, the indicating mark 19a is eliminated from the movement indicating part 19C. However, the thickness of the movement indicating part 36 is a dimension such that the surface of the movement indicating part 36 does not come out to the outside of the cover member 32.

The shielding part 35 covers openings on the base end side (the side opposite to the grasping part 14) of each step hole portion 33a and the elongate hole 33 in the surface of the cover member 32, and a straight shielding end 35a orthogonal to the movement direction of the movement indicating part 36 is formed on the tip end side of the step hole portion 33a and the elongate hole 33.

The material of the shielding part 35 is appropriately a material that is opaque such that the movement indicating part 36 cannot be seen through. In the present embodiment, a metallic thin plate is bonded or welded on the cover member 32.

The position of the shielding end 35a is made to coincide with the outer edge portion of the movement indicating part 36 (refer to the two-dot chain line of FIGS. 7A and 7B) nearest to the tip end side when the grasping part 14 is exactly closed.

For this reason, as shown in FIG. 7B, only while the grasping g part 14 is open, a portion of the movement indicating part 36 protrudes from the shielding end 35a to the tip end side, and can be confirmed by the operator Op. In that case, the protruding amount of the movement indicating part 36 from the shielding end 35a represents the magnitude of the opening angle of the grasping part 14.

Additionally, the overall movement indicating part 36 is covered and hidden with the shielding part 35 while the grasping part 14 is closed.

For this reason, the movement indicating part 36 constitutes an indicating member that advances and retracts from the inside of the shielding part 35 to the outside thereof, interlocking with the movement of the connection rotation shaft 19A of the connecting member 19 that is a coupling member.

In this way, according to the treatment section 10A of the present modified example, the operator Op can comprehend the opening angle of the grasping part 14 by viewing the protruding amount of the movement indicating part 36 from the shielding end 35a of the shielding part 35. When the movement indicating part 36 is in a state where the movement indicating part substantially touches the step hole portion 33a on the tip end side, the protruding amount of the movement indicating part 36 becomes the maximum. For this reason, the operator Op can observe the ratio of the movement indicating part 36 that accounts for an opening portion formed by the step hole portion 33a and the shielding part 35 in an analog manner, thereby comprehending a margin up to the opening angle or maximum opening angle of the grasping part 14.

Additionally, the operator can comprehend that grasping part 14 is closed in a case where the movement indicating part 36 cannot be seen.

Additionally, in the present modified example, a situation where the grasping force increases from a state where the grasping part 14 is closed cannot be comprehended because the movement of the movement indicating part becomes invisible. However, in a case where the grasping force is increased after the grasping part 14 grasps a grasp target, an increase in the grasping force can be comprehended by observing a reduction in the protruding amount from the shielding end 35a.

In this way, in the present modified example, the opening and closing amount or force application condition of the grasping part 14 of the treatment section 10A can be easily comprehended while viewing the treatment section 10A during manipulation.

### Second Modified Example

Next, a second modified example of the present embodiment will be described.

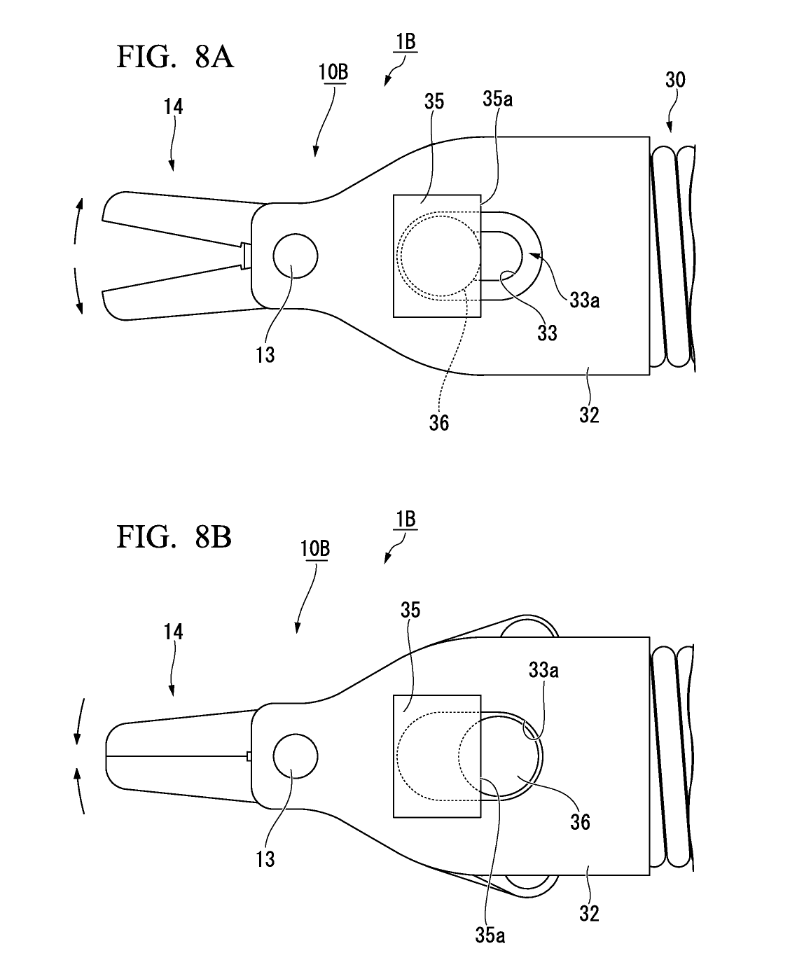

FIG. 8A is a schematic front view showing a state where a treatment section of a medical treatment tool of a second modified example of the first embodiment of the present invention is opened. FIG. 8B is a schematic front view showing a state where the treatment section of the medical treatment tool of the second modified example of the first embodiment of the present invention is closed.

A treatment tool 1B (medical treatment tool) of the present modified example, as shown in FIG. 8A, includes a treatment section 10B instead of the treatment section 10A of the first modified example, and can be mounted on the slave arms 200a to 200d and used as the treatment tools 240a to 240d of the medical manipulator system shown in FIG. 1 similarly to the first modified example.

Differences from the first modified example will mainly be described below.

The treatment section 10B has a configuration in which the arrangement position of the shielding part 35 of the treatment section 10A of the first modified example is changed to a position on the tip end side so as to exactly cover and hide the movement indicating part 36 when the movement indicating part 36 had moved nearest to the tip end side in each step hole portion 33a.

That is, the shielding part 35 in the present modified example covers the overall movement indicating part 36 (refer to a broken line of FIG. 8A) when the grasping part 14 is opened at the maximum, and the shielding end 35a coincides with the position of the outer edge portion of the movement indicating part 36 nearest to the base end side.

For this reason, as shown in FIG. 8B, in a case where the grasping part 14 is opened at an angle smaller than the maximum opening angle, a portion of the movement indicating part 36 protrudes from the shielding end 35a to the base end side depending on an opening angle, and can be confirmed by the operator Op. In that case, the protruding amount of the movement indicating part 36 from the shielding end 35a represents the magnitude of the opening angle of the grasping part 14.

Additionally, in the present modified example, the movement indicating part 36 moves to the base end side of the step hole portion 33a after the grasping part 14 is closed. Therefore, it is also possible to comprehend this amount of movement as a change in the protruding amount from the shielding end 35a.

In this way, according to the treatment section 10B of the present modified example, the operator Op can comprehend an increase in the grasping force of the grasping part 14 by viewing the projection amount of the movement indicating part 36 from the shielding end 35a of the shielding part 35. The maximum protruding amount of the movement indicating part 36 is in a state where the movement indicating part 36 substantially touches the step hole portion 33a on the tip end side. For this reason, the operator Op can observe the ratio of the movement indicating part 36 that accounts for an opening portion formed by the step hole portion 33a and the shielding part 35 in an analog manner, thereby comprehending a margin up to the opening angle of the grasping part 14 or the increase limit of the grasping force in an analog manner.

Additionally, the operator can comprehend that the opening angle of the grasping part 14 is the maximum in a case where the movement indicating part 36 is not confirmed.

In this way, in the present modified example, the opening and closing amount or force application condition of the grasping part 14 of the treatment section 10B can be easily comprehended while viewing the treatment section 10B during manipulation.

In the present modified example, when the movement indicating part 36 has moved nearest to the tip end side in each step hole portion 33a, the arrangement position of the shielding part 35 is a position where the movement indicating part 36 is exactly covered and hidden. The present invention is not limited thereto, and for example, when the movement indicating part 36 has moved to a position (for example, a position where a needle is grasped) where the grasping part 14 has started grasping, the arrangement position of the shielding part 35 may be a position where the movement indicating part 36 is exactly covered and hidden so that it can be seen how much load is applied depending on a protruding amount.

### Third Modified Example

Next, a third modified example of the present embodiment will be described.

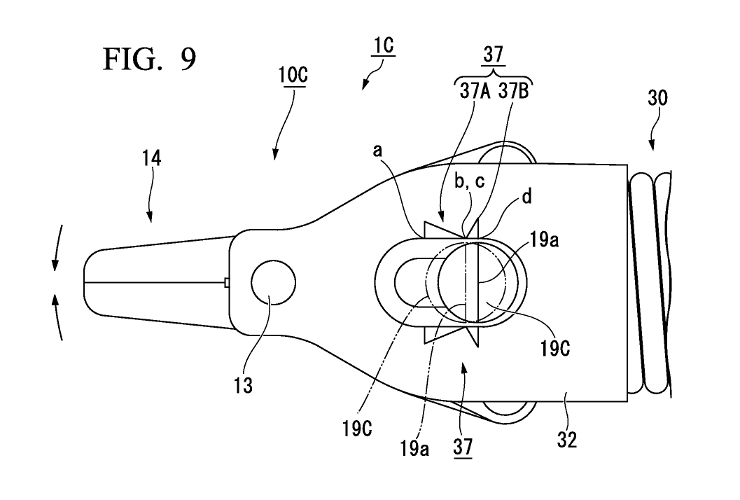

FIG. 9 is a schematic front view showing a treatment section of a medical treatment tool of the third modified example of the first embodiment of the present invention.

A treatment tool 1C (medical treatment tool) of the present modified example, as shown in FIG. 9, includes a treatment section 10C instead of the treatment section 10 of the treatment tool 1 of the first embodiment, and can be mounted on the slave arms 200a to 200d and used as the treatment tools 240a to 240d of the medical manipulator system shown in FIG. 1 similarly to the first embodiment.

Hereinafter, differences from the first embodiment will mainly be described.

The treatment section 10C includes a scale mark 37 (a reference index part or reference scale) instead of the graduation lines 34 of the treatment section 10 of the first embodiment.

The scale mark 37 is a reference scale for referring to the amount of relative movement of the movement indicating part 19C to the cover member 32.

The scale mark 37 includes triangular marks 37A and 37B that are formed in a pair at positions that faces each other in the lateral direction of the step hole portion 33a, in the surface of the cover member 32.

The triangular mark 37A is a right-angled triangle in which a side sandwiched by a right angle having a vertex a and one acute angle having a vertex b is arranged in the longitudinal direction of the step hole portion 33a. The vertex a is arranged so as to become the position of the maximum open position graduation 34a in the first embodiment and the vertex b is arranged so as to become the position of the basis closed position graduation 34b.

The triangular mark 37B is a right-angled triangle in which a side sandwiched by a right angle having a vertex d and one acute angle having a vertex c is arranged in the longitudinal direction of the step hole portion 33a. The vertex c is arranged so as to become the position of the basis closed position graduation 34b in the first embodiment and the vertex d is arranged so as to become the position of the grasping force reference auxiliary graduation 34d. For this reason, the vertex c coincides with the vertex b of the triangular mark 37A.

From such a configuration, the indicating mark 19a being located in the region of the triangular mark 37A is the same as the indicating mark 19a being located between the maximum open position graduation 34a and the basis closed position graduation 34b in the first embodiment.

Additionally, the indicating mark 19a being located in the region of the triangular mark 37B is the same as the indicating mark 19a being located between the basis closed position graduation 34b and the grasping force reference auxiliary graduation 34d in the first embodiment.

Additionally, in the triangular mark 37A, since an oblique side inclines in a direction separating from the step hole portion 33a as it goes from the vertex b to the vertex a, a change in the height of the oblique side at a position shown by the indicating mark 19a shows that the opening angle of the grasping part 14 is gradually increasing.

Additionally, in the triangular mark 37B, since an oblique side inclines in the direction separating from the step hole portion 33a as it goes from the vertex c to the vertex d, the height of the oblique side at a position shown by the indicating mark 19a shows that the grasping force of the grasping part 14 is gradually increasing.

Accordingly, the operator Op can refer to the vertex of the scale mark 37 or a change in the height of the oblique side by viewing the display 900a for an operator to thereby visually comprehend the amount of relative movement or movement position of the indicating mark 19a of the movement indicating part 19C.

Accordingly, similarly to the first embodiment, the operator Op can exactly comprehend the opening angle of the grasping part 14 without keeping his/her eyes off the display 900a for an operator on which the manipulation target and the grasping part 14 are displayed. Therefore, manipulation can be smoothly continued while monitoring the manipulation target even by delicate manipulation.

The graduation lines 34 of the first embodiment are an example of discrete scales that are easy to gradually comprehend the amount of relative movement, whereas the scale mark 37 of the present modified example is an example of an analog scale that is easy to visually comprehend the amount of relative movement.

### Fourth Modified Example

Next, a fourth modified example of the present embodiment will be described.

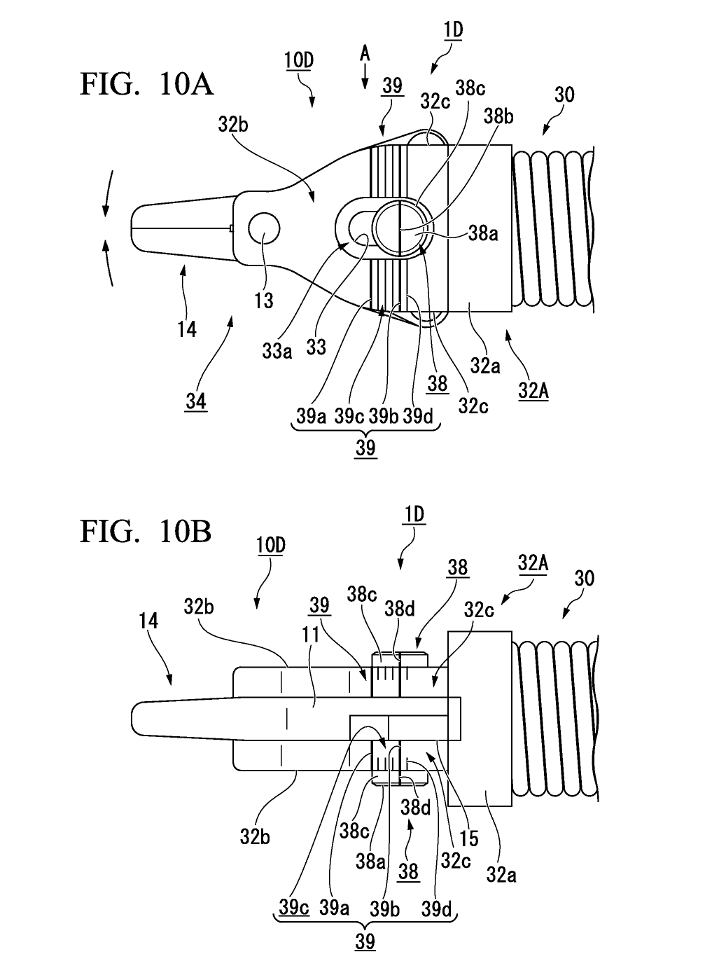

FIG. 10A is a schematic front view showing a treatment section of a medical treatment tool of the fourth modified example of the first embodiment of the present invention. FIG. 10B is a side view seen at A of FIG. 10A.

A treatment tool 1D (medical treatment tool) of the present modified example, as shown in FIGS. 10A and 10B, includes a treatment section 10D instead of the treatment section 10 of the treatment tool 1 of the first embodiment, and can be mounted on the slave arms 200a to 200d and used as the treatment tools 240a to 240d of the medical manipulator system shown in FIG. 1 similarly to the first embodiment.

Hereinafter, differences from the first embodiment will mainly be described.

The treatment section 10D is including a cover member 32A (substrate), graduation lines 39 (a reference index part or reference scales), and a movement indicating part 38, instead of the cover member 32, the graduation lines 34, and the movement indicating part 19C of the treatment section 10 of the first embodiment.

In the cover member 32A, a tip end (side where the grasping part 14 is provided) of the cover member 32 of the first embodiment is formed by flat-plate-shaped tip end cover portions 32b that face each other in parallel with a toggle mechanism interposed therebetween, and a base end side is formed by a cylindrical base end cover portion 32a to which the sheath part 30 can be connected.

As for the shape of the tip end cover portion 32b in plan view, as shown in FIG. 10A, a plate-shaped portion of the same width as the base end cover portion 32a extends to a position approximately equal to a straight portion in the longitudinal direction of the elongate hole 33 from the base end side to the tip end side, is narrowed as it goes further toward the tip end, and has a constant width in the vicinity of the forceps rotation shaft 13.

For this reason, planar side surface portions 32c including a plane parallel to the longitudinal direction of the elongate hole 33 are formed on the side surfaces of each tip end cover portion 32b on the base end side.

The graduation lines 39, as shown in FIG. 10A and FIG. 10B, include a maximum open position graduation 39a, a basis closed position graduation 39b (closed state starting position reference index), an open position auxiliary graduation 39c, and a grasping force reference auxiliary graduation 39d (closing force reference index), which are a parallel line group that is obtained by extending the maximum open position graduation 34a, the basis closed position graduation 34b, the open position auxiliary graduation 34c, and the grasping force reference auxiliary graduation 34d in the graduation lines 34 of the first embodiment to the planar side surface portions 32c, respectively.

However, the open position auxiliary graduation 39c and the grasping force reference auxiliary graduation 39d have a narrow line width so as to be capable of being visually distinguished from the maximum open position graduation 39a and the basis closed position graduation 39b. Colors may be changed or mutual line types may be changed so as to be more easily distinguished visually.

As the method of forming the graduation lines 39, the same method as the graduation lines 34 may be adopted.

Each movement indicating part 38 is formed in a shape such that a tip end face 38a and a portion of the side surface 38c protrude sideways from the tip end cover portion 32b by changing the thickness of the movement indicating part 19C of the first embodiment, and is formed in a disc shape in which the ridgeline of the tip end face 38a is chamfered.

The tip end face 38a of each movement indicating part 38 is formed with a straight indicating mark 38b that is the same as the indicating mark 19a. Both ends of the indicating mark 38b are formed with straight indicating marks 38d that extend in the direction of generatrices of the side surfaces 38c. For this reason, the indicating marks 38b and 38d are aligned on the same plane.

From such a configuration, according to the treatment section 10D, the movement of the indicating mark 38b of the tip end face 38a of each movement indicating part 38 is confirmed by the operator Op with the movement of the connection rotation shaft 19A if the tip end cover portion 32b is seen. For this reason, the amount of relative movement or movement position of the indicating marks 38b can be comprehended by referring to the graduation lines 39 on the tip end cover portion 32b.

Additionally, the movement of the indicating mark 38d of the side surface 38c of each movement indicating part 38 is confirmed by the operator Op with the movement of the connection rotation shaft 19A if the planar side surface portion 32c is seen. For this reason, the amount of relative movement or movement position of the indicating marks 38d can be comprehended by referring to the graduation lines 39.

In this way, in the present modified example, if either each tip end cover portion 32b or each planar side surface portion 32c that constitutes the periphery of the treatment section 10D is displayed on the display 900a for an operator, the operator Op can comprehend the amount of relative movement and movement position of the movement indicating part 38.

As a result, even if the orientation of the treatment section 10D is changed by manipulation, a possibility that any of the indicating marks 38b and 38d of the movement indicating part 38 and any of the graduation lines 39 is displayed on the display 900a for an operator becomes very high. Accordingly, since the opening angle or force application condition of the grasping part 14 can be comprehended without changing a visual field region on the display 900a for an operator, manipulation can be smoothly continued while monitoring a manipulation target at a position that is easy to see.

### Fifth Modified Example

Next, a fifth modified example of the present embodiment will be described.

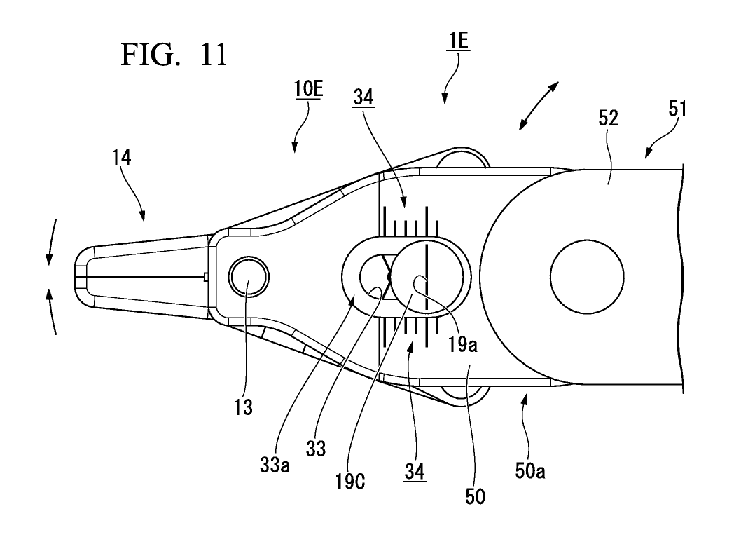

FIG. 11 is a schematic front view showing a treatment section of a medical treatment tool of the fifth modified example of the first embodiment of the present invention.

A treatment tool 1E of the present modified example, as shown in FIG. 11, includes a treatment section 10E and a robot joint 51 that rotationally drives the treatment section 10E instead of the treatment section 10 of the treatment tool 1 of the first embodiment, and can be mounted on the slave arms 200a to 200d and used as the treatment tools 240a to 240d of the medical manipulator system shown in FIG. 1 similarly to the first embodiment.

Hereinafter, differences from the first embodiment will mainly be described.

In the treatment section 10E, the sheath part 30 of the treatment section 10 of the first embodiment is eliminated, and a cover member 50 that has a joint coupling portion 50a coupled to a joint portion 52 of the robot joint 51 is included instead of the cover member 32.

The robot joint 51 has a coupling portion to be coupled to the slave arms 200a to 200d at a base end (not shown) thereof, and has the joint portion 52 to be coupled with the joint coupling portion 50a of the treatment section 10E provided at a tip end shown in FIG. 11.

The joint portion 52 can rotate the treatment section 10E within the paper of FIG. 11 by wire manipulation (not shown) or the like in the present modified example.

The treatment section 10E of the present modified example is an example in a case where the treatment tool includes the joint portion of robot joint 51 or the like, and the effects by the action of the movement indicating part 19C and the graduation lines 34 is the same as those of the first embodiment.

### Second Embodiment

Next, a second embodiment of the present invention will be described.

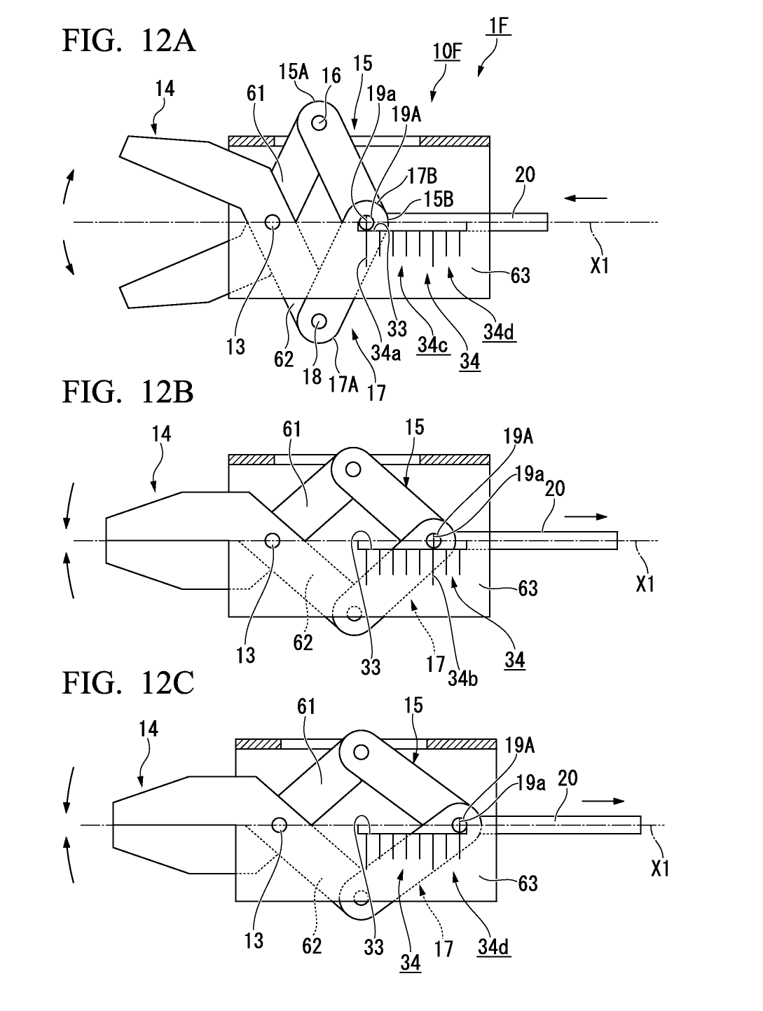

FIG. 12A shows a state where a treatment section of a medical treatment tool of the second embodiment of the present invention is opened. FIG. 12B shows a state where the treatment section of the medical treatment tool of the second embodiment of the present invention is closed. FIG. 12C is a schematic partial cross-sectional view showing a state where the treatment section of the medical treatment tool of the second embodiment of the present invention is closed and a grasping force is increased.

A treatment tool 1F (medical treatment tool) of the present embodiment, as shown in FIG. 12A, includes a treatment section 10F instead of the treatment section 10 of the treatment tool 1 of the first embodiment, and can be mounted on the slave arms 200a to 200d and used as the treatment tools 240a to 240d of the medical manipulator system shown in FIG. 1 similarly to the first embodiment.

Hereinafter, differences from the first embodiment will mainly be described.

In the treatment section 10F, the opening and closing of the grasping part 14 of the treatment section 10 of the first embodiment is performed by a pantagraph mechanism instead of a toggle mechanism, and the movement indicating part is modified.

For this reason, the treatment section 10F includes a first forceps piece 61 (treatment tool piece), a second forceps piece 62 (treatment tool pieces), and a cover member 63 (substrate) instead of the first forceps piece 11, the second forceps piece 12, and the cover member 32 of the treatment section 10 of the first embodiment, and the movement indicating part 19C is eliminated.

The first forceps piece 61 and the second forceps piece 62 are a pair of forceps pieces (treatment tool pieces) that constitute the grasping part 14, and have the same tip ends as the first forceps piece 11 and the second forceps piece 12, respectively, and are mutually rotatably coupled by the forceps rotation shaft 13 in a state where the first and second forceps pieces are bent in a direction opposite to the first forceps piece 11 and the second forceps piece 12 on the base end side and are crossed in the shape of the letter X at central portions.

Additionally, an end of the first forceps piece 61 on the base end side is rotatably coupled with the tip end 17A of the link member 17 via the link rotation shaft 18.

Additionally, an end of the second forceps piece 62 on the base end side is rotatably coupled with the tip end 15A of the link member 15 via the link rotation shaft 16.

The base ends 15B and 17B of the link members 15 and 17 are coupled with the manipulating member 20 by the connecting member 19 (not shown), similarly to the first embodiment.

However, since the pantagraph mechanism is constituted in the present embodiment, the position of the connection rotation shaft 19A is located closer to the base end side (the side opposite to the forceps rotation shaft 13) than the link rotation shafts 16 and 18, in the advance and retraction axis of the base ends 15B and 17B.

In the cover member 63, the cover member 32 of the first embodiment is modified to a shape that can accommodate the pantagraph mechanism of the present embodiment, and the step hole portion 33a is eliminated.

For this reason, the graduation lines 34 of the present embodiment are provided to make a pair at positions that face each other in the lateral direction of the elongate hole 33 along the longitudinal direction of the elongate hole 33.

Additionally, although the graduation lines 34 includes the maximum open position graduation 34a, the basis closed position graduation 34b, the open position auxiliary graduation 34c, and the grasping force reference auxiliary graduation 34d similarly to the first embodiment, the positions of the respective graduations, the intervals between the graduations, and the number of the graduations are appropriately changed on the basis of the movement range of the connection rotation shaft 19A of the present embodiment.

Additionally, in the present embodiment, the connection rotation shaft 19A extends within the elongate hole 33, and the indicating mark 19a is formed at the end of the connection rotation shaft 19A so that the amount of movement with respect to the cover member 63 is visually recognized from the outside of the cover member 63. That is, the end of the connection rotation shaft 19A constitutes the movement indicating part in the present embodiment.

Additionally, although illustration is omitted, the sheath part 30 is connected to the base end of the cover member 63 similarly to the treatment tool 1 of the first embodiment.

From such a configuration, in the treatment section 10F, as shown in FIG. 12A, the grasping part 14 that protrudes from the tip end side of the cover member 63 is opened by advancing the manipulating member 20 to the tip end side. At this time, if the connection rotation shaft 19A moves to the tip end side at the maximum in the elongate hole 33, the indicating mark 19a of the connection rotation shaft 19A moves to a position that that is aligned with the maximum open position graduation 34a in the graduation lines 34.

Additionally, the grasping part 14 is closed by retreating the manipulating member 20 to the base end side. As shown in FIG. 12B, in a state where the grasping part 14 is exactly closed and a grasping force is not generated in the grasping part 14, the indicating mark 19a moves to a position that is aligned with the basis closed position graduation 34b in the graduation lines 34.

In the state where the indicating mark 19a is located between the maximum open position graduation 34a and the basis closed position graduation 34b, the opening angle of the grasping part 14 changes according to the distance of the indicating mark 19a from the basis closed position graduation 34b. The operator Op can comprehend the opening angle of the grasping part 14 by comprehending the position of the indicating mark 19a with reference to the open position auxiliary graduation 34c.

If the manipulating member 20 is further retracted to the base end side as shown in FIG. 12C, the first forceps piece 61 and the second forceps piece 62 elastically deform, and the grasping part 14 is pressurized. As a result, the grasping force increases. In that case, the operator Op can comprehend the grasping force applied to grasping part 14 by comprehending the position of the indicating mark 19a with reference to the grasping force reference auxiliary graduation 34d.

In this way, even if the opening and closing mechanism of the grasping part 14 is changed to the pantagraph mechanism, according to the treatment section 10F of the treatment tool 1F, the connection rotation shaft 19A and the graduation lines 34 that are movement indicating parts are provided at the cover member 63 of the treatment section 10F so as to be confirmed by the operator Op from the outside. Therefore, the opening and closing amount or force application condition of the grasping part 14 of the treatment section 10F can be easily comprehended while viewing the treatment section 10F during manipulation.

### Third Embodiment

Next, a third embodiment of the present invention will be described.

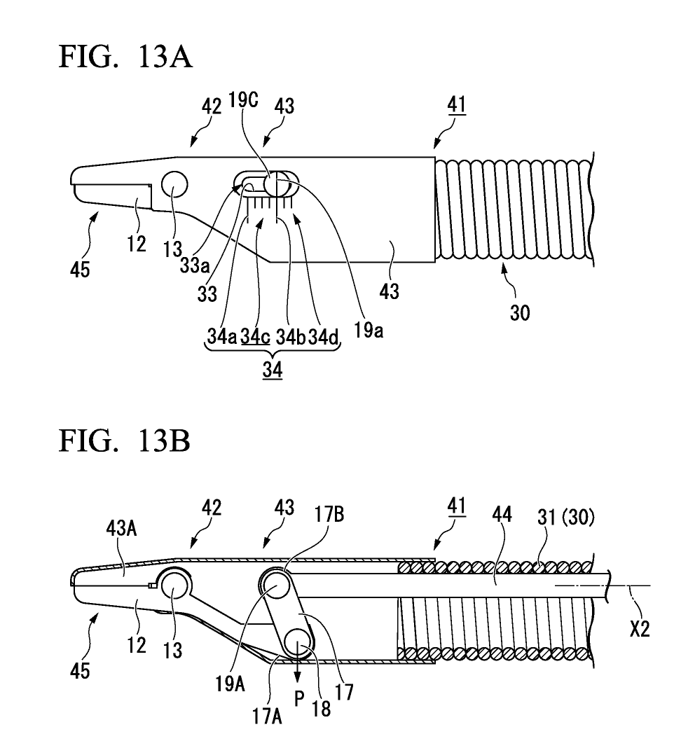

FIG. 13A is a schematic front view showing a treatment section of a medical treatment tool of a third embodiment of the present invention. FIG. 13B is a schematic cross-sectional view showing the treatment section of the medical treatment tool of the third embodiment of the present invention.

A treatment tool 41 of the present embodiment, as shown in FIGS. 13A and 13B, is different from the treatment tool 1 of the first embodiment in that only one of the pair of forceps pieces is rotatable. The treatment tool 41, similarly to the first embodiment, can be mounted on the slave arms 200a to 200d and used as the treatment tools 240a to 240d of the medical manipulator system shown in FIG. 1.

Hereinafter, differences from the first embodiment will mainly be described.

The treatment tool 41, as shown in FIG. 13B, has a cover member 43 (substrate) that has a forceps piece portion 43A that is equivalent to a first forceps piece of a pair of forceps pieces, and only the second forceps piece 12 is made rotatable around the forceps rotation shaft 13. Therefore, the treatment tool does not include the link member 15 and the link rotation shaft 16.

In the forceps piece portion 43A, and the second forceps piece 12, a portion that protrudes to the tip end side of the cover member 43 closer to the tip end side than the forceps rotation shaft 13 constitutes the grasping part 45 that is opened and closed by the rotation of the second forceps piece 12.

The manipulating member 44 is a rod having predetermined rigidity, and has a tip end coupled with the link member 17 via the connection rotation shaft 19A. The positional relationship between the tip end 17A and the base end 17B of the link member 17 in a state where the forceps piece portion 43A and the second forceps piece 12 are brought into contact with each other and are closed, the coupling length of the link member 17, or the like is set approximately similarly to the treatment tool 1.

The elongate hole 33 and the step hole portion 33a are provided in the cover member 43, along the advance and retraction direction of the connection rotation shaft 19A similarly to the first embodiment. The maximum retraction amount of the manipulating member 44 is set to a value such that the second forceps piece 12 or the link member 17 deforms plastically or the connection rotation shaft 19A does not move closer to the base end side than the link rotation shaft 18.

Additionally, in the surface of the cover member 43, the graduation lines 34 including the maximum open position graduation 34a, the basis closed position graduation 34b, the open position auxiliary graduation 34c, and the grasping force reference auxiliary graduation 34d are formed at the side of the step hole portion 33a in the lateral direction like the first embodiment.

However, as for the graduation lines 34 of the present embodiment, for example, the positions of the respective graduations, the intervals between the graduations, and the number of the graduations are appropriately changed on the basis of the movement range of the movement indicating part 19C of the present embodiment in the toggle mechanism of the present embodiment.

If the treatment tool 41 configured in this way advances the manipulating member 44 similarly to the treatment tool 1, as shown in FIG. 7, the grasping part 45 is opened. Then, by further performing the manipulation input of retracting the manipulating member 44 after the grasping part 45 is closed, the grasping force generated in the grasping part 45 can be increased.

Here, the reaction force P of the output Fo that acts on the link rotation shaft 18 acts on the manipulating member 44 in a direction in which the connection rotation shaft 19A is separated from the axis X2 of the manipulating member 44. Since the predetermined rigidity of the manipulating member 44 is set to such a degree that the manipulating member does not deform against the maximum reaction force within the manipulation range of the manipulating member 44, the connection rotation shaft 19A moves along the axis X2 of the manipulating member 44 against the reaction force even when the grasping force is increased.

In this way, also in the treatment tool 41 of the present embodiment, by performing the manipulation input of retracting the manipulating member similarly to the treatment tool 1 of the first embodiment, a grasping force generated in the grasping part can be efficiently increased even by a relatively small manipulation input.

Additionally, the movement indicating part 19C and the graduation lines 34 act similarly to the treatment tool 1 of the first embodiment. That is, since the operator Op can comprehend the amount of movement of the indicating mark 19a with reference to the graduation lines 34 by viewing the display 900a for an operator, the operator can easily comprehend the opening angle of the grasping part 45 without keeping his/her eyes off the display 900a for an operator on which the manipulation target and the grasping part 45 are displayed, or can easily comprehend the degree of an increase in the grasping force during closing manipulation. Therefore, manipulation can be smoothly continued while monitoring the manipulation target even by delicate manipulation.

Although the respective embodiments and modified examples of the present invention have been described above, the technical scope of the present invention is not limited to the above embodiments and modified examples, and without departing from the scope of the present invention, various changes or omissions can be made to respective constituent elements or constituents of the respective embodiments can be combined together.

For example, although an example in which the pair of treatment tool pieces is supported by the common rotation shaft has been described in the above respective embodiments and respective modified examples, the pair of forceps pieces may be supported by different rotation shafts, respectively. In this case, by dividing the rotation centers of the pair of treatment tool pieces, the angle β shown in FIG. 6 can be made smaller, and the output Fo generated in the grasping part can be improved.

Additionally, in the treatment section in the treatment tool of the embodiments (including modified examples) of the present invention, the amount of a force in a direction in which the pair of treatment tool pieces is closed is not necessarily increased.

For example, the following configuration example can be given.

One end of a pair of treatment tool pieces is rotatably coupled by the forceps rotation shaft 13, and the other end forms an openable and closable structure. An intermediate portion between one end and an other end of each treatment tool piece are provided with the link rotation shafts 16 and 18, and the tip ends 15A and 17A of the link members 15 and 17 are connected to the link rotation shafts 16 and 18 similarly to the first embodiment. Additionally, the base ends 15B and 17B of the link members 15 and 17 are connected to the connecting member 19 via the connection rotation shaft 19A, and the manipulating member 20 is fixed to the connecting member 19. In that case, the connection rotation shaft 19A is located between the link rotation shafts 16 and 18 and the forceps rotation shaft 13, and the manipulating member 20 extends toward the forceps rotation shaft 13 side.

According to such a configuration, the pair of treatment tool pieces are opened and closed by advancing and retracting the manipulating member 20, and when the manipulating member 20 is advanced to the link rotation shaft 16 and 18 side, the tip end (other end) of each treatment tool piece is opened outward, and the amount of a force in the opening direction can be increased by the action of the toggle mechanism.

Such a configuration can be favorably used as, for example, a treatment tool that opens the pair of treatment tool pieces and pushes out a living body tissue by inserting the treatment section into a gap between living body tissues or the like and advancing the manipulating member 20.

Additionally, an example in which the amount of movement of the movement indicating part is confirmed from the outside by exposing at least a portion of the movement indicating part accommodated in the elongate hole or the step hole portion to the outside of the substrate has been described in the description of the above respective embodiments and respective modified examples. However, a configuration may be adopted in which the movement indicating part is covered with, for example, a transparent cover or the like if a situation in which the movement indicating part moves is confirmed from the outside.

Additionally, an example in which the basis closed position graduation 34b is provided at a position where the grasping part is closed with a grasping force of 0 when the grasping part does not grasp a body to be grasped has been described in the description of the above respective embodiments and respective modified examples. However, a position with a predetermined opening angle can be adopted as a basis position, and graduations can be provided so that this basis position can be seen.

According to such a configuration, manipulation becomes easy and is convenient in a case where the size or shape of the body to be grasped is determined or the opening angle when the grasping of the grasping part is started is determined.

Additionally, an example in which the elongate hole 33 has both the functions of a movement guide of the connection rotation shaft 19A and a stopper that regulates the movement range of the connection rotation shaft 19A has been described in the description of the above respective embodiments and respective modified examples. However, as long as the movement track and movement range of the connection rotation shaft 19A can regulate by other means, the elongate hole 33 may be provided as a hole portion that allows the connection rotation shaft 19A to be inserted therethrough without contacting the connecting rotation shaft.

For example, if a configuration is adopted in which the rigidity of the manipulating member is appropriately set, and for example, the sheath part or the like also serves as the movement guide of a manipulating guide, the movement guide or stopper of the connection rotation shaft 19A can be omitted, and the elongate hole 33 and the connection rotation shaft 19A can be configured so as not to come into contact with each other.

In this case, since there is no sliding between the connection rotation shaft 19A and the elongate hole 33, the manipulation force during advance and retraction can be reduced, and sliding wear can also be reduced.

Additionally, an example in which the treatment tool is provided at a manipulator in a medical manipulator system has been described in the description of the above respective embodiments and respective modified examples. However, the treatment tool of the present invention is not limited to an aspect in which the treatment tool is connected to the manipulator, and can also be used as a treatment tool that is not connected to the manipulator.

Additionally, an example in which the movement indicating parts and the reference index parts are provided in two or more places has been described in the description of the above respective embodiments and respective modified examples. However, as long as an operator can make visual recognition during manipulation, the movement indicating part and the reference index part may be provided in one place only, respectively.

Additionally, in the above respective embodiments and respective modified examples, mechanisms that can indicate the movement indicating part and the reference index part in an enlarged manner, for example, lenses, may be provided at those portions. Thereby, visibility is improved, and even in a treatment tool with fine diameter, the opening and closing amount or force application condition of the treatment section can be easily comprehended.

Additionally, in the description of the third modified example of the above first embodiment, an example in which shape changes in an analog manner like the triangular marks 37A and 37B has been described as an example in which the reference scales are analog scales. However, as other analog scales, color bars in which colors or the concentration of a color change in a given direction may be adopted.

Additionally, a case where the indicating mark 19a is formed at the end of the connection rotation shaft 19A and the connection rotation shaft 19A also serves as the movement indicating part has been described in the description of the above second embodiment. However, this modified example can be applied irrespective of whether the link members constitute a toggle mechanism or a pantagraph mechanism. Accordingly, a configuration in which the indicating mark 19a is provided at the end of the connection rotation shaft 19A instead of the movement indicating part 19C may be adopted in the above first embodiment. Additionally, a configuration using the movement indicating part 19C may be adopted in the above second embodiment.

Other modified examples hereinafter obtained by combining the constituent elements of the above respective embodiments and respective modified examples will be described.

For example, a configuration may be adopted in which the graduation lines 34 in the above first embodiment are added onto the cover member and the shielding part 35 of the above first and second modified examples. In this case, it is possible to more precisely comprehend the protruding amount of the movement indicating part 36.

Additionally, for example, the following modified example can be configured by combining the above first modified example and the above second modified example.

In this modified example, the width of the shielding part 35 is adjusted to the width of the movement indicating part 19C, and when the movement indicating part 19C has moved to an appropriate basis position, for example, a position where the indicating mark 19a is aligned with the basis closed position graduation 34b in the first embodiment, the shielding part is installed at a position where the movement indicating part 19C is exact covered.

According to such a modified example, the protruding amount corresponds to the opening angle of the grasping part 14 if the movement indicating part 19C protrudes to the tip end side and is seen. Additionally, the protruding amount corresponds to an increase in the grasping force of the grasping part 14 if the movement indicating part 19C protrudes to the base end side and is seen.

While preferred embodiments of the present invention have been described, the present invention is not limited to the embodiments.

Additions, omissions, substitutions, and other variations may be made to the present invention without departing from the spirit and scope of the present invention. The present invention is not limited by the above description, but by the appended claims.

1. A medical treatment tool comprising:
a treatment section which has a pair of treatment tool pieces, at least one of the pair of treatment tool pieces being rotatably supported with respect to a substrate;
a manipulating member which rotates the pair of treatment tool pieces, and opens and closes the pair of treatment tool pieces by advancing and retracting in an axis direction of the manipulating member with respect to the substrate;
a link member which has a first end coupled to at least one of the pair of treatment tool pieces and has a second end coupled to the manipulating member;
a coupling member which couples the link member and the manipulating member at the second end;
a movement indicating part which moves interlocking with a movement of the coupling member in an advance and retraction direction of the manipulating member and is provided so that an amount of movement with respect to the substrate is visually recognized from an outside of the substrate; and
a reference index part which is provided at the substrate and refers to a amount of relative movement of the movement indicating part to the substrate.

2. The medical treatment tool according to claim 1,
wherein the substrate is provided with a through hole portion,
wherein the movement indicating part is provided at the coupling member that is movably inserted through the through hole portion, and
wherein the reference index part is a reference scale provided around the through hole portion, or on the through hole portion.

3. The medical treatment tool according to claim 1,
wherein the reference index part has a shielding part that covers a portion of the surface of the substrate,
wherein the movement indicating part has an indicating member that advances and retracts from an inside of the shielding part to an outside and that interlocks with the movement of the coupling member, and
wherein the amount of movement of the movement indicating part is indicated depending on an amount of an advance and retraction of the indicating member from the shielding part.

4. The medical treatment tool according to claim 1,
wherein the reference index part includes
a closed state starting position reference index that allows a position of the movement indicating part when a state where the pair of treatment tool pieces is closed is started to be referred to, and
a closing force reference index that allows a change in the position of the movement indicating part corresponding to an increase in a closing force between the pair of treatment tool pieces in a state where the pair of treatment tool pieces is closed to be referred to.

5. The medical treatment tool according to claim 1,
wherein an advance and retraction axis of the second end is parallel to the axis of the manipulating member,
wherein a distance between the advance and retraction axis of the second end and the first end is shorter than the length of the link member, and
wherein a length when a line segment connecting the second end and a rotation center of the pair of treatment tool pieces is projected on the advance and retraction axis is shorter than a length when a line segment connecting the first end and the rotation center is projected on the advance and retraction axis.

6. A manipulator comprising: the medical treatment tool according to claim 1.
